(19) United States
(12) Patent Application Publication
(10) Pub. No.: US 2021/0260600 A1
(43) Pub. Date: Aug. 26, 2021

(54) Automated device and method to purify biomaterials from a mixture by using magnetic particles and disposable product-contact materials

(71) Applicant: Sunil Mehta, Morrisville, NC (US)

(72) Inventor: Sunil Mehta, MORRISVILLE, NC (US)

(21) Appl. No.: 17/163,472

(22) Filed: Jan. 31, 2021

Related U.S. Application Data
(60) Provisional application No. 62/969,064, filed on Feb. 1, 2020.

(51) Int. Cl. B03C 1/28; B01L 3/00; G01N 33/543

(52) U.S. Cl. CPC B03C 1/284; B01L 3/5085; G01N 33/54326; B01L 2200/0647; B03C 2201/26; B01L 2200/025; B03C 2201/18

(57) ABSTRACT

This invention relates to a device and method of using the device for purification that separates material of interest from contaminating materials using non-porous magnetic particles and single-use or disposable materials that come in contact with the material of interest. The process encompasses multiple cycles in a single batch to reduce the cost of magnetic particles. This method can be executed in a fully automated manner by a controller that manages different inputs and outputs of system hardware.

### RELATED APPLICATIONS

The present application claims priority from U.S. Provisional Patent Application No. 62/969,064, filed Feb. 1, 2020, and from U.S. Provisional Patent Application No. 63/002,319, filed Mar. 30, 2020, the disclosures of which are hereby incorporated herein by reference in their entireties.

### FIELD OF THE INVENTION

The present invention is related to methods, and systems that are used for separating macromolecules, including biomaterials, such as cells, cellular components, proteins, lipids, carbohydrates and tissues from a mixture.

### BACKGROUND

Biotherapeutics have revolutionized healthcare globally. Biotherapeutics are generally complex molecules or particles as compare to traditional chemical-based simple molecule drugs. Many of the biotherapeutics are recombinant proteins, like antibodies, as they can have a very high affinity for therapeutic targets, fewer side effects, lower immunogenic rejection, and long half-life. Some other biotherapeutics include viruses, bacteria, whole cells or parts of cells (for example, exosomes), carbohydrates, lipids, DNA, or RNA.

Biotherapeutics typically cost higher to manufacture as compared to chemical-based drugs. Much of their manufacturing cost is attributed to complex downstream purification processes¹.

Recombinant proteins can be produced using a variety of host expression systems, including bacteria, yeast, and mammalian cells. Recombinant antibodies are generally produced in mammalian cells as some other host systems lack sophisticated post-translational machinery that is required to produce functional antibodies.

Due to the complexity of proteins, they are recombinantly produced in host organisms either intracellularly (e.g. bacterial host expression systems) or secreted extracellularly (e.g. yeast and mammalian expression systems). When mammalian expression systems are used to produce proteins, the cell culture harvest is clarified by separating cells from the supernatant using centrifugation and/or filtration technologies. Harvest clarification requires expensive equipment and/or single-use filter modules, large manufacturing floor space, a large volume of buffers, and expensive utilities. Also, harvest clarification adds at least one additional day to the processing time. An increase in cell density in cultures improves productivity, which can reduce the overall cost of therapeutics. At the same time, increase in cell density poses several challenges for traditional harvest clarification technologies. Disc-stack centrifuges are commonly used for harvest clarification to clarify cell culture and discharge unwanted cells in the form of a slurry. During this process, some of the product is also lost in the slurry but it is not a huge problem if the cell density is low as the number of discharge cycles is limited. If the cell concentration increases, product loss also increases as the number of discharge cycles increases with increasing cell density. Also, disc-stack centrifuges exert high shear force on cells, causing the release of host cell proteins and proteolytic enzymes in the clarified harvest². This not only creates downstream purification challenges but also negatively impacts product quality. Filtration based technologies also become impractical for high cell density cultures as they require very large systems and surface areas while causing significant loss of product and high levels of host cell protein contamination².

High cell density not only poses challenges for upstream processes but also downstream processes as much larger chromatography columns are required for purification and host cell protein removal becomes challenging, Current chromatography technologies rely on porous beads to create large chromatography surface area with a relatively smaller volume. Due to the very small size of pores in beads, the penetration of the molecules to be purified requires significant diffusion time leading to long processing time. The chromatography columns packed with these beads also require high-pressure feeding of liquids as the flow resistance is higher due to small pore channels in the beads. In addition, each cycle requires a cleaning and regeneration step as the smaller pore size retains some of the non-specific materials after retrieval of the product of interest. This not only slows down the processing but also requires large amounts of additional buffers for washing and regeneration of chromatography resin. If the affinity ligands are sensitive to cleaning agents, the chromatography material can only be used for one cycle. Also, therapeutics or vaccine macromolecules that are larger in size (e.g. viruses, bacteria, whole cells, or exosomes) than the pores in the chromatography resin, cannot be purified using current chromatography processes.

Most chromatography systems use stainless-steel equipment that requires cleaning between batches to reduce cross-contamination. The cleaning validation of systems is expensive and adds significant delays between batches. In addition, cleaning process requires significant infrastructure and once installed, the equipment becomes immobile as it is hard piped. Single-use systems have gained traction in recent years due to their lower capital requirements, flexibility, reduced turn-around times, no-risk of cross-contamination between batches, and environmental friendliness. Although most of the single-use chromatography systems use disposable product contact surfaces, the column is still reused for multiple batches due to its cost. Simulated moving bed technology has tried to make the columns disposable by increasing the number of cycles at the cost of adding additional processing time. However, as all these systems use porous chromatography media/resin, the challenges related to porous chromatography resin remain.

Purified and unpurified biomaterials from bodily fluids are also valuable and used for many different purposes. For example, immunoglobulins purified from donated human plasma (intravenous immunoglobulin or IVIG) are used to treat several disorders including life-threatening sepsis. Factor VIII purified from donated human plasma is used to treat hemophilia in factor VIII deficient patients. Immunoglobulins purified from animals immunized with toxins are used to treat poisoning from venomous snake bites. In the absence of a vaccine or therapeutic during a pathogenic outbreak, convalescent sera from a recovered healthy donor can be used as a therapeutic to treat critical patients or as a prophylactic treatment for the population at risk (Ref. 3-4).

There are some drawbacks to using the convalescent serum. Generally, newly recovered donors are not able to donate large amounts of serum and can easily go into hypovolemic shock. In addition, donated unpurified sera can potentially harm the receiving critical patients as it contains many biomaterials (including infectious agents) other than therapeutic immunoglobulins (Ref. 5). For example, viruses can unintentionally be transmitted from donor's serum to recipients. Also, immunological reactions from serum such as serum sickness are common (Ref. 6).

During serum donations, a limited amount of biomolecule of interest is collected from each donor as they cannot donate more than a certain amount (unless they are sacrificed). In many applications of plasma (including factor VIII, IVIG, and anti-venom), the plasma from several donors is shipped to a processing facility, pooled, and biomaterials of interest are isolated. This process usually takes months to complete and if the goal is to combat a novel pathogen during an epidemic or a pandemic, the pathogen may mutate during that time and the purified therapeutic may not be effective.

### SUMMARY

The method described in this invention solves many problems associated with the purification processes of biotherapeutics. This method significantly reduces the total processing time and simplifies downstream processing by eliminating or reducing the need for clarification before chromatography. All product contact surfaces are suitable for biotherapeutics manufacturing (e.g. meet USP class VI requirements) and are replaced after each batch but can support multiple cycles of processing within the same batch. The method described here uses particles that are attracted to magnets (ferromagnetic, paramagnetic, or superparamagnetic), have large surface area due to their smaller size, are preferably non-porous, and have specific binding properties. These particles exhibit very short residence time for binding as most of the binding sites are on the surface. The size of these particles is from 20-10,000 nm, which is up to 20,000 times smaller than the standard porous chromatography resin beads and the surface is modified to have properties such as affinity for a specific type of molecules (e.g. modification with Protein A ligand for antibodies purification or modification with antibodies that specifically bind a biomaterial), positive or negative charge, hydrophobic interactions, or two or more of these properties. The particles may be referred as magnetic particles or beads in this invention.

These particles are mixed in with starting material which could be cell culture or partially clarified broth. An external magnetic field is applied to recover these particles from cell culture. If the magnetic susceptibility is low due to smaller size of magnetic particles, a high gradient magnetic field is used to isolate particles mixed in with cell culture. In our challenging testing conditions with a mixture of Fe3O4 particles (300 nm; cubic shaped) and high concentration of Saccharomyces cerevisiae in media, >99% of the particles were captured after application of a high gradient magnetic field generated by a magnetic field ranging from 0.1-1.7 Tesla. Small-scale batch chromatography methods to purify proteins (in microgram or nanogram scale) using magnetic beads are currently available but those mostly use porous magnetic beads that are not recycled and are operated manually. Commercially available magnetic particles are spherical with a diameter that is equal to or greater than 1 micron and used only for a single cycle at a very small scale (e.g. Sera-Mag from GE Healthcare Life Sciences and Dynabeads from ThermoFisher Scientific). These particles are not well suited for the current invention due to their porosity, large size, and recycling capabilities.

The magnetic particles are chosen in this invention have a high surface area to volumetric ratio. For example, the surface area to volumetric ratio of tetrahedron and cubic particles is at least 25% greater than the ratio of spherical particles. The attachment of ligand to the surface of magnetic particles is achieved by a linker that can bind to the ligand and the inorganic surface of magnetic particles. In one embodiment, linkers with silane groups are used to chemically bond with magnetic particles. The other end of the linker is chosen such that it can readily bind to the ligand that is used for purification. For example, the epoxide group at the other end of the linker can react with common primary amine, sulfhydryl, or carboxyl group that are commonly found on different amino acids of proteins. 3-Glycidoxypropyl trimethoxysilane or 3-Glycidoxypropyl triethoxysilane are two examples of such linkers. In our studies, 0.5-3% w/w treatment with 3-Glycidoxypropyl trimethoxysilane provided excellent conjugation to Fe3O4 cubic particles of 300 nm average size.

During affinity chromatography, macromolecules of interest bind to the magnetic particles that have a surface affinity for those macromolecules. The recovered magnetic particles can be washed multiple times to remove impurities and then elution is performed by introducing an elution buffer that dissociates the binding of the macromolecules of interest from the magnetic particles. In one embodiment, a low pH buffer is used to dissociate antibodies that are captured by Protein A coupled magnetic particles. Similarly, imidazole is used to elute histidine-tagged protein bound to Ni-NTA particles. The eluted macromolecules flow through while magnetic particles are retained due to the external magnetic field. If the affinity performance of particles is reduced after a certain number of cycles, due to nonspecific occlusion of specific binding sites by impurities, the particles can be regenerated using regeneration solutions (e.g. NaOH, chaotropic agents: Urea, Guanidine).

Flow-through chromatography reduces impurities by binding the impurities to magnetic particles and letting the macromolecules of interest flow through. The surface of magnetic particles is modified such that they have an affinity for impurities but not for the macromolecules of interest. These impurities could be DNA, host cell proteins, or other unwanted macromolecules. In one embodiment, positive-charged quaternary ammonium magnetic particles can be used to remove contaminating negative-charged DNA from the protein of interest. The contaminating impurities bind to magnetic particles while macromolecules of interest flow through and are collected. After each cycle, magnetic particles are regenerated to remove the bound contaminants so that magnetic particles can be reused for subsequent cycles.

### BRIEF DESCRIPTION OF THE DRAWINGS

FIG. 1 depicts an example of an affinity purification method.

FIG. 2 depicts an overview of the process to purify immunoglobulins from human blood.

FIG. 3 depicts an example to purify biomaterials from bodily fluids.

### DETAILED DESCRIPTION OF THE INVENTION

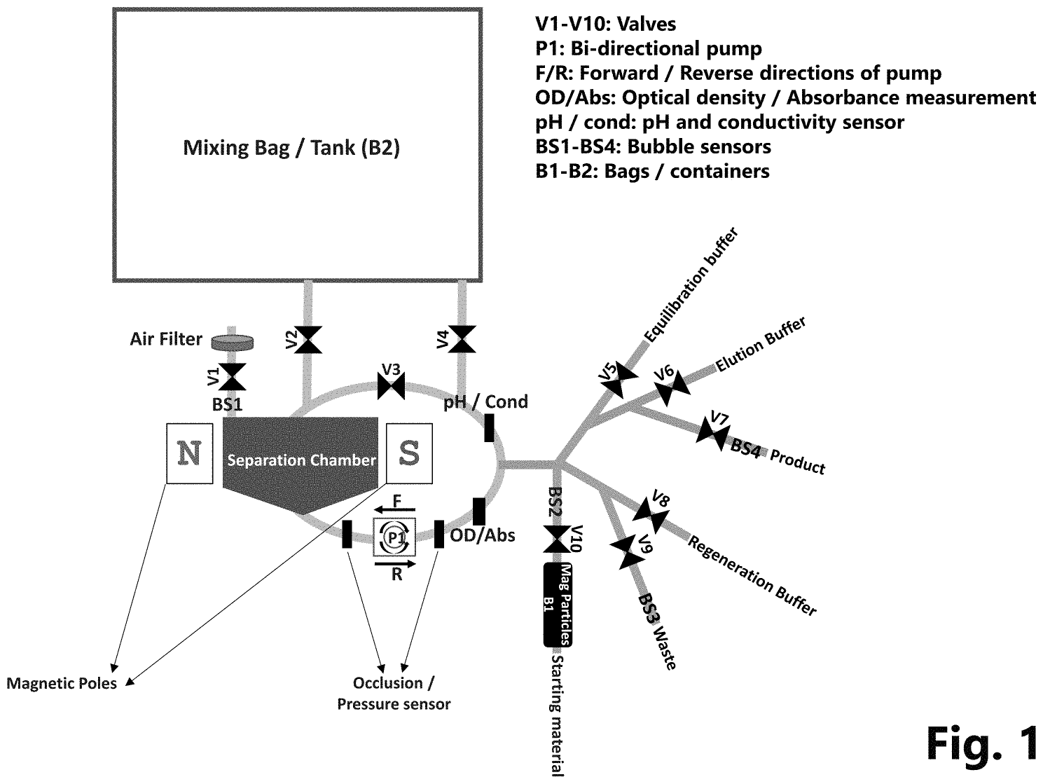

The binding of macromolecules or impurities (for flow-through mode) is performed in a mixing container or a bag in which the magnetic particles are mixed in with starting solution containing a mixed population of macromolecules and impurities (e.g. cell culture or partially clarified cell culture). Mixing is performed to improve the binding kinetics. The mixing can be performed by employing standard mixing technologies or by recirculation of fluid via a single pump or multiple pumps (e.g. P1 in FIG. 1). After the binding of macromolecules to the magnetic particles is complete, the magnetic field is introduced via either switching on electromagnets or physically moving permanent magnets closer to the binding chamber. To capture smaller particles with low magnetic susceptibility, a high gradient magnetic field can be generated by employing ferromagnetic stainless-steel wool or other ferromagnetic support material inside the separation chamber. The magnetic particles are retained while the remaining material is pumped out of the system into waste. The retention efficiency of the magnetic particles can again be increased by employing standard mixing technologies (e.g. rotating impeller) or by simple recirculation of fluid via a single pump or multiple pumps. Mixing not only increases the retention efficiency but also provides homogeneous capture of magnetic particles on a ferromagnetic support material. In the absence of mixing the magnetic particles can easily clog the ferromagnetic support material and can cause overpressure. If overpressure is detected by the pressure sensor, a fluid path is created such that (e.g. V3 opens with remaining valves closed) P1 recirculates the liquid within the separation chamber without a magnetic field and then the magnetic field is gradually increased so that particles are uniformly captured in the separation chamber.

During affinity purification, the bound particles are washed either in the presence or absence of a magnetic force to remove any nonspecifically bound impurities. The bound macromolecules are eluted from the magnetic particles by the introduction of elution buffer to the separation chamber in the presence or the absence of an external magnetic force. If the binding performance of particles reduces after multiple cycles, the particles can be cleaned using a sanitization buffer (e.g. urea or guanidine hydrochloride). The whole cycle can be repeated multiple times until a complete batch is purified.

In case of flow-through purification, macromolecules of interest do not bind (e.g. recombinant proteins) to the magnetic particles and flow through while the impurities (e.g. DNA) are captured by the magnetic particles. Magnetic particles can be regenerated using a sanitization buffer for additional cycles.

In each cycle, a specific volume of starting material (out of a batch) is pumped into the system. This is called the cycle volume and is calculated based on the amount of magnetic particles that are loaded into the system, the estimated amount of material to be purified (product or impurity amount), the desired number of cycles or the total processing time, and recycling capability of magnetic particles. For example, if Protein A magnetic particles with a capacity of 50 g/kg (gram of antibody captured per kg of magnetic particles) are used to affinity purify a batch of 2000 L with 5 g/L antibody concentration, a single cycle will require 200 kg of magnetic particles. On the other hand, if the magnetic particles are recycled for 50 cycles, only 4 kg of magnetic particles are required. The time required to run 50 cycles will be around 50 times more than to run a single cycle, but the savings realized from using a lesser amount of magnetic particles and smaller size of the equipment, greatly outweighs the longer processing time. If a single cycle takes 10 minutes, 50 cycles will take just over 8 hours, which is a reasonable processing time for a 2000 L batch as traditional methods require 2-4 days of processing.

A cylindrical separation chamber with a conical bottom (with the apex at the bottom) is used to isolate magnetic particles during different parts of the process. In another embodiment, the top of the separation chamber is conical with Apex at the top. The separation chamber is fabricated out of materials that are poorly attracted by a magnetic force (e.g. LDPE, high durometer Silicone, polypropylene, polystyrene, EVA, PVC etc.) and may contain steel wool or wire mesh to enhance the magnetic field via high gradient magnetic field generation, to capture magnetic particles that are small and have weak magnetic susceptibility. In one embodiment, the material of steel wool or wire mesh (1 micron-1000 micron) is ferromagnetic stainless-steel (e.g. 430 or 410 stainless steel) to provide corrosion resistance. The ferromagnetic steel wool can also be coated with metals (e.g. Nickel or Chromium) or plastics to substantially reduce or eliminate corrosion. In one embodiment, separation efficiency and throughput of the process is increased by placing multiple separation chambers in parallel and/or in series. Multiple separation chambers increase processing flow rates significantly due to parallel processing. When the separation chambers are placed in series, they improve retention of particles significantly at higher flow rates.

An external magnetic field, generally in the range of 0.1-1.7 Tesla is applied to the separation chamber by either moving permanent magnets to the proximity or turning on an electromagnet surrounding the chamber or by other means. As required in the purification process, the controller controls the magnetic force applied to the separation chamber by turning it on or off or by reducing or increasing its strength.

The complete disposable manifold including tubing, separation chamber (with or without steel wool), mixing bag, pH/conductivity flow cell, optical density and absorbance measurement flow cell, and filter is pre-assembled and is generally sterilized before use and is capable of withstanding multiple cycles in a batch. Tubing or pipes that have single-use product contact surfaces are used to circulate different fluids through the system. For example, flexible silicone, C-flex, or PVC tubing can be used to connect different parts of the system to channel fluids. A disposable manifold is connected to bags or vessels containing magnetic particle slurry, different buffers, starting material (e.g. bioreactor), and final purified material via connectors or welding of thermoplastic tube. The connectors can be aseptic or non-aseptic.

Product contact surfaces that come in contact with the material of interest, include the inner surfaces of tubes, connectors, all flow-cells (e.g. pH/conductivity, OD etc.), pressure sensor, and the separation chamber. The Product contact surfaces also include outer surface of the particles.

A controller with HMI (human machine interface) is used by the user to automatically or manually control and receive information from parts of the system, which include pumps (speed, direction, start/stop, feedback etc.), valves (open, close, feedback), occlusion/pressure sensors, optical density and absorbance detector, bubble sensors, in-line pH/conductivity meter, temperature sensor, and magnetic-field control system. This information is provided as analog or digital signals to and from the controller. Commercially automation systems from various vendors such as Siemens, Backhoff, or Allen-Bradley are available as complete solutions that include controller, input/output digital or analog cards, and HMI. Examples are Siemens SIMATIC S7-1200 kit or Backhoff TwinCAT 3 automation platform.

The user inputs various process parameters into a process screen that is part of the HMI. Process parameters include cycle volume, incubation, mixing, wash, and recirculation times, pH, conductivity, and optical density and absorbance values to start or stop a step. Once the user starts the process by providing input to the HMI, the controller can follow the process screen and automatically run the process using entered process parameters. Multiple valves are controlled (FIG. 1; V1-V10) to form different fluid paths during different phases of the purification process. In one embodiment, pinch valves are used to open and close the fluid path within a flexible tube.

The present invention now is described more fully hereinafter with reference to the accompanying drawings, in which some embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “include”, “including”, “comprises”, and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Fluids can be liquid or air. Embodiments with liquids may also be applicable to air.

The scheme shown in FIG. 1 is an example to introduce various fluids at different process steps during the purification process. Two examples of automated methods to purify biotherapeutics and an example of an automated method to purify from bodily fluids are described below. In the affinity purification method, the material of interest binds to the particles while in the flow-through purification method, the material of interest flows-through and the material bound to the particles is generally discarded.

### Example 1: Affinity Purification Method

In the FIG. 1, V1-V10 are the valves, P1 indicates a bi-directional pump, FIR indicates forward/reverse directions of the pump, OD/Abs indicates Optical Density/absorbance measurement, pH/coed indicates pH and conductivity sensor, BS1-4 indicates bubble sensors, N or S indicates poles of a magnet, and B1-B2 indicates bags or containers.

Different steps for the affinity purification process are detailed below. The pump is turned off and all valves are closed at the end of each step. All flexible bags or containers containing different liquids are connected via tubing as per FIG. 1. A bag or container holding the magnetic particles is attached to the tube connected to V10. Starting material is not connected to the system at this stage.

Step 1. Loading of Magnetic Particles:

After the disposable has been loaded into the system, V1 and V10 are opened while the remaining vales remain closed. P1 is turned on to move magnetic particles in liquid suspension from the container or flexible bag B1 to the separation chamber. Once the bubble sensor BS2 detects air (due to emptying of B1), the pump is stopped after the remaining material in the tubing between B1 and P1 has entered the separation chamber. Thereafter V3 is opened, V1 and V10 are closed and P1 is turned on to recirculate material in the separation chamber. At the same time, the magnetic force is turned on to capture magnetic particles as they recirculate through the loop. The capture of the magnetic particles by the separation chamber can be monitored by measuring optical density and absorbance that is specific for the particles, by the OD sensor (e.g. at 370 nm). The flow rate of the pump can be reduced to allow better capture of magnetic particles. Once the magnetic particles are captured, P1 is reversed, V3 is closed, and V1 and V10 are opened to fill B1 with liquid in which magnetic particles were suspended. The B1 bag can be manually shaken to suspend magnetic particles that may be left in the bag. Once BS2 detects bubbles, the pump P1 direction is switched to forward to move the liquid back into the separation chamber. Once BS2 detects bubbles, V10 is closed and V3 is opened to recirculate again in the loop until optical density or absorbance drops substantially. The flow rate of the pump can be reduced to allow better capture of magnetic particles. At this stage, the supernatant is discarded by reversing the pump direction and opening V9 and closing V3. The pump is stopped after bubble sensor BS3 detects air.

Step 2. Washing of Magnetic Particles with Magnetic Field:

After V9 is closed, the equilibration buffer is introduced by opening V5 and V1 and running the pump in the forward direction until BS1 detects liquid. Equilibration buffer is used to wash the particles and to reduce non-specific binding to the particles. Phosphate Buffered Saline (PBS) and Tris based buffers are examples of an equilibration buffer. At that stage, V3 is opened and V5 is closed. Washing progress can be assessed by monitoring data from inline pH/conductivity sensors. Generally, after less than a minute of washing the magnetic particles in this manner, pump direction is reversed, V9 is opened, and V3 is closed. When bubble sensor BS3 detects air, V3 is opened momentarily to allow the emptying of the remaining liquid in the loop. This process of washing can be repeated 1-2 times.

Cleaning/Regeneration Step:

Whenever magnetic particles require regeneration, sanitization, or robust cleaning, sanitization or cleaning buffer connected to V8 can be used. The magnetic field is turned off and the equilibration buffer is introduced by opening V8 and V1 and running the pump in the forward direction until BS1 detects liquid. At that stage, V3 is opened and V8 is closed. After recirculation of the magnetic particles to create mixing, the magnetic field is turned on to collect magnetic particles in the separation chamber. The flow rate of the pump can be reduced to allow better capture of magnetic particles. Once the optical density is reduced substantially, the pump direction is reversed, V9 is opened, and V3 is closed. When bubble sensor BS3 detects air, V3 is opened momentarily to allow emptying of the remaining liquid in the loop. At last, the pump is stopped, and all valves are closed. The regeneration step is generally followed by Step 5 or Step 2 to remove traces of regeneration buffer from the system.

Step 3. Binding of Magnetic Particles to Macromolecules:

The B1 bag is removed and starting material source is attached to the tube connected to V10. The starting material source could be a bioreactor or a bag or vessel containing the starting material. Starting material is introduced into the system by opening V1 and V10, turning off the magnetic field, and running the pump in the forward direction until BS1 detects liquid. At this stage V1 is closed, V2 is opened, the pump is continued to tun in the forward direction for a set time until the total volume of starting material that is equal to the cycle volume has been loaded into the system. If the mixing tank B2 is a rigid container, an air vent with 0.2 micron filter is attached to the top of the container to displace the air by liquid coming in. Once the set amount of liquid equal to the cycle volume has been pumped in, the V4 is opened and V10 and V1 are closed to mix magnetic beads with starting material. The flow direction can be switched back and forth to improve mixing. Once the incubation time required for the binding of macromolecules to the beads is complete, the magnetic field is turned on to capture the magnetic beads in the separation chamber. The flow rate of the pump can be reduced to allow better capture of magnetic particles. Most of the beads can be captured by this recirculation process and capture efficiency can be measured by optical density sensor. The pump direction is then reversed, V4 is closed and V9 is opened to capture the remaining magnetic particles and to discard the starting material (without macromolecule of interest) to waste. When BS3 detects air, V2 is closed and V3 and V4 are opened for only a couple of seconds to allow emptying of the remaining liquid in the loop.

Step 4. Washing of Magnetic Particles with Magnetic Field:

Step 2 from above is followed.

Step 5. Washing of Magnetic Particles without Magnetic Field:

V9 is closed, the magnetic field is turned off, and the equilibration buffer is introduced by opening V5 and V1 and running the pump in the forward direction until BS1 detects liquid. At that stage, V3 is opened and V5 is closed. After recirculation of the magnetic particles to create mixing, the magnetic field is turned on to collect magnetic particles in the separation chamber. The flow rate of the pump can be reduced to allow better capture of magnetic particles. Once the OD370 or an optical density that specifically detects the particles, is reduced substantially, the pump direction is reversed, V9 is opened, and V3 is closed. When bubble sensor BS3 detects air, V3 is opened momentarily to allow the emptying of the remaining liquid in the loop. This process of washing is repeated 1-2 times.

Step 6. Elution:

Step 6.1: V9 is closed, V6 and V1 are opened, and the pump is operated for short time in forward direction to fill the tube up to the pump. Then V6 is closed, V9 is opened, and the pump is reversed. This step cleans tubes with elution buffer.

Step 6.2: To fill the separation chamber with elution buffer, the magnetic field is turned off and the elution buffer is introduced by opening V6 and running the pump in the forward direction until BS1 detects liquid. At that stage, V3 is opened and V6 is closed. After recirculation of the magnetic particles to create mixing, the magnetic field is turned on to collect magnetic particles in the separation chamber. The flow rate of the pump can be reduced to allow better capture of magnetic particles. Once the OD to detect magnetic particles is reduced substantially and OD to detect eluted material (e.g. OD at 280 nm for proteins) is maximal, V7 is opened, V3 is closed, and pump direction is reversed to run until BS4 detects air. To empty the remaining liquid in the loop, V3 is opened momentarily while the pump is running and then V3 and V7 are closed. This elution process is repeated from step 6.2 for 1-2 times to collect eluted molecules. When this process is repeated, the step to clean the tubes with elution buffer (Step 6.1) is not required.

After step 6, steps 2-7 are repeated until the end of the bioreactor is detected by air in the BS2 sensor. At that point the last cycle is completed by running steps 2-7 and the disposable assembly can be discarded.

### Example 2: Flow-Through Purification Method

In the FIG. 1, V1-V10 are the valves, P1 indicate a bi-directional pump, FIR indicates forward/reverse directions of the pump, OD/Abs indicates Optical Density/absorbance measurement, pH/cond indicates pH and conductivity sensor, BS1-4 indicates bubble sensors, N or S indicates poles of a magnet, and B1-B2 indicates bags or containers.

Different steps for the flow-through purification process are detailed below. The pump and all valves are closed at the end of each step. All flexible bags or containers containing different liquids are connected via tubing as per FIG. 1. A bag or container holding the magnetic particles is attached to the tube connected to V10. Starting material is not connected to the system at this stage.

Step 1. Loading of Magnetic Particles:

After the disposable has been loaded into the system, V1 and V10 are opened while the remaining vales remain closed. P1 is turned on to move magnetic particles in liquid suspension from the container or flexible bag B1 to the separation chamber. Once the bubble sensor BS2 detects air (due to emptying of B1), the pump is stopped after the remaining material in the tubing between B1 and P1 has entered the separation chamber. Thereafter V3 is opened, V1 and V10 are closed and P1 is turned on to recirculate material in the separation chamber. At the same time, the magnetic force is turned on to capture magnetic particles as they recirculate through the loop. The capture of the magnetic particles by the separation chamber can be monitored by measuring optical density and absorbance that is specific for the particles, by the OD sensor (e.g. at 370 nm). Once the magnetic particles are captured, P1 is reversed, V3 is closed, and V1 and V10 are opened to fill B1 with liquid in which magnetic particles were suspended. The B1 bag can be manually shaken to suspend magnetic particles that maybe left in the bag. Once BS2 detects bubbles, the P1 direction is switched to forward direction to move the liquid back into the separation chamber. Once BS2 detects bubbles, V10 is closed and V3 is opened to recirculate again in the loop until optical density or absorbance drops substantially. At this stage, the supernatant is discarded by reversing the pump direction and opening V9 and closing V3. The pump is stopped after bubble sensor BS3 detects air.

Step 2. Washing of Magnetic Particles with Magnetic Field:

After V9 is closed; the equilibration buffer is introduced by opening V5 and V1 and running the pump in the forward direction until BS1 detects liquid. At that stage, V3 is opened and V5 is closed. Washing progress can be assessed by monitoring data from inline pH/conductivity sensors. After less than a minute of washing the magnetic particles in this manner, the pump direction is reversed, V9 is opened, and V3 is closed. When bubble sensor BS3 detects air, V3 is opened momentarily to allow the emptying of the remaining liquid in the loop. This process of washing can be repeated 1-2 times.

Cleaning/Regeneration Step:

Whenever magnetic particles require regeneration, sanitization, or robust cleaning, sanitization or cleaning buffer connected to V8 can be used. The magnetic field is turned off and the equilibration buffer is introduced by opening V8 and V1 and running the pump in the forward direction until BS1 detects liquid. At that stage, V3 is opened and V8 is closed. After recirculation of the magnetic particles to create mixing, the magnetic field is turned on to collect magnetic particles in the separation chamber. The flow rate of the pump can be reduced to allow better capture of magnetic particles. Once the optical density specific to the particles is reduced substantially, the pump direction is reversed, V9 is opened, and V3 is closed. When bubble sensor BS3 detects air, V3 is opened momentarily to allow the emptying of the remaining liquid in the loop. At last, the pump is stopped, and all valves are closed. The regeneration step is generally followed by Step 6 or Step 2 to remove regeneration buffer traces from the system.

Step 3. Binding of Magnetic Particles to Impurities:

Starting material is introduced into the system by opening V10, turning off the magnetic field, and running the pump in the forward direction for a set time while V2 is opened. If the mixing tank is a rigid container, an air vent with a 0.2 micron filter is attached to the top of the container to displace the air by liquid coming in. Once the set amount of liquid equal to the cycle volume has been pumped in, the V4 is opened and V10 is closed to mix magnetic beads with starting material. Once the incubation time required for the binding of impurities to the beads is complete, the magnetic field is turned on to capture the magnetic beads in the separation chamber. Lowering the flow rate of the pump allows better capture of magnetic particles. The majority of the beads can be captured by this recirculation process. The pump direction is then reversed, V4 closed and V7 is opened to capture the remaining magnetic particles and to collect the purified material. When BS4 detects air, V2 is closed and V3 is opened momentarily to allow emptying of remaining liquid in the loop.

Step 4. Washing of Magnetic Particles with Magnetic Field:

Washing with equilibrium buffer can be used to improve yield by recovering the leftover starting material from the fluid path. This step can be eliminated to shorten the total processing time. The equilibration buffer is introduced by opening V5 and V1 and running the pump in the forward direction until BS1 detects liquid. At that stage, V3 is opened and V5 is closed. After less than a minute of washing the magnetic particles in this manner, the pump direction is reversed, V7 is opened, and V3 is closed. When bubble sensor BS4 detects air, V3 is opened momentarily to allow the emptying of the remaining liquid in the loop.

Step 5. Cleaning/Regeneration:

Magnetic particles are regenerated to remove the bound impurities by washing with regeneration, sanitization, or cleaning buffer that is connected to V8. The magnetic field is turned off and regeneration buffer is introduced by opening V8 and V1 and running the pump in the forward direction until BS1 detects liquid. At that stage, V3 is opened and V8 is closed. After recirculation of the magnetic particles to create mixing, the magnetic field is turned on to collect magnetic particles in the separation chamber. The flow rate of the pump can be reduced to allow better capture of magnetic particles. Once the optical density specific to the particles (e.g. OD370) is reduced substantially, pump direction is reversed, V9 is opened, and V3 is closed. When bubble sensor BS3 detects air, V3 is opened momentarily to allow the emptying of the remaining liquid in the loop.

Step 6. Washing of Magnetic Particles without Magnetic Field:

The magnetic field is turned off and the equilibration buffer is introduced by opening V5 and V1 and running the pump in the forward direction until BS1 detects liquid. At that stage, V3 is opened and V5 is closed. After recirculation of the magnetic particles to create mixing, the magnetic field is turned on to collect magnetic particles in the separation chamber. The flow rate of the pump can be reduced to allow better capture of magnetic particles. Once the optical density specific to the particles (e.g. OD370) is reduced substantially, pump direction is reversed, V9 is opened, and V3 is closed. When bubble sensor BS3 detects air, V3 is opened momentarily and closed to allow the emptying of the remaining liquid in the loop. At last, the pump is stopped, and all valves are closed. Washing progress can be assessed by monitoring data from inline pH/conductivity sensors. This process of washing is repeated 1-2 times.

After step 6, steps 3-6 are repeated until the end of bioreactor is detected by air in the BS2 sensor. At that point the last cycle is completed by running steps 3-6 and the disposable assembly can be discarded.

### Example 3: Method to Purify Biomaterials from Bodily Fluids

The device and method of using the device described in this invention are not only useful for biomanufacturing but also enable the purification of biomaterials from bodily liquids of animals or humans. In the device and method described here, biomaterials of interest can be purified at the donation site and the remaining material can be returned to the donor after capturing the biomaterial of interest so that this process can be repeated multiple times to purify much larger amounts of biomaterials of interest in a single donation. This method eliminates the risk of a hypovolemic shock for the donor. Also, this significantly reduces processing, shipping, storage, and logistics costs over the existing methods. Another advantage of the method is that blood and other bodily fluids can directly be used without requiring any pre-processing (e.g. removal of cells etc.).

Multiple cycles of blood collection allow purification of significantly higher amounts of immunoglobulins that can treat multiple recipients without causing hypovolemic shock to the donor. Another advantage of this new method of purification is that recipients of the purified material have a significantly lower risk as compared to the convalescent serum. This device and process have the potential to save millions of human lives in an epidemic or a pandemic. This process can be used to manage current and emerging strains of SARS-CoV-2 as antibodies purified from donations will be against the most recent propagating strains of the virus and can be injected in a short timeframe to the recipients who are exposed to the same strain of the virus. The isolated immunoglobulins from convalescent sera can be screened against pathogens to identify neutralizing antibodies and this information can be used in developing recombinant antibodies and vaccines.

In several disorders, harmful or toxic biomaterials accumulate in patient's blood. This method can be used to target those harmful or toxic biomaterials and reduce their circulating concentration. For example, in Rheumatoid Arthritis, Psoriatic Arthritis, Ankylosing Spondylitis, Plaque Psoriasis, Crohn's Disease, and Ulcerative Colitis, the Tumor Necrosis Factor (TNF) concentration is elevated. Several antibody-drug treatments are available that target TNF but after repeated dosing, most patients develop neutralizing antibodies against those drugs requiring them to switch to a different drug. This method can also be used to reduce circulating TNF by specifically capturing and removing TNF from blood to ameliorate the condition. Since no external drug is infused into the patients, patients do not suffer from the drug side effects and this method of treatment can effectively be used lifelong by them. This process is similar to the flow-through purification process.

Similar to the effects of repeated dosing of anti-TNF antibody therapeutics, repeated dosing of other protein therapeutics also leads to the generation of neutralizing antibodies against the administered protein therapeutic that reduces their effectiveness over time. In contrast, this method of treatment can effectively be used lifelong by patients.

In another embodiment, this method is used to remove excess drugs from blood to reduce their side effects. For example, a high dose of a drug for the treatment of cancer or other indications is needed to have a maximal effect. After a drug has specifically bound to the target, any free drug may remain circulating in the blood, causing side effects. This technology can be used to specifically remove unbound or free drug remaining in the blood to reduce its side-effects.

The device uses a single-use or disposable kit and particles with specific properties. All product contact surfaces in a single-use kit are suitable for the medical device (e.g. meet USP class VI requirements) and can be replaced after each donation but can support multiple cycles of processing within the same donation. For donations from animals, the disposable kit can potentially be re-used for more than one donation after a wash cycle with sanitization and equilibration buffer to sanitize and wash single-use surfaces that come in contact with bodily fluids. The method described here uses particles that are attracted to magnets (e.g. ferromagnetic, paramagnetic; or superparamagnetic); have large surface area due to their smaller size, are preferably non-porous, and have specific binding properties. These particles exhibit very short residence time for binding as most of the binding sites are on the surface. The size of these particles is from 10-10000 nm, which is up to 20000 times smaller than the standard porous chromatography resin beads. The surface of these particles is modified with ligands that have specific binding properties (e.g. surface attachment of Protein A for antibody purification, or attachment of antibodies that have an affinity for a specific biomaterial, or attachment of positive or negative charged ligands, or attachment of hydrophobic ligands, or ligands with mixed properties).

The bodily fluids containing target biomaterial are drawn either directly or indirectly in a bag containing these particles that have an affinity for the targeted biomaterial and are mixed. Other buffers or reagents can be added to the collection bag for conditioning. For example, citrate or heparin can be added to the collection bag to manage blood clotting. An external magnetic field is applied to recover these particles from bodily fluids. If the magnetic susceptibility is low due to the smaller size of magnetic particles, a high gradient magnetic field is used to isolate particles mixed in with bodily fluids. Small-scale batch chromatography methods to purify proteins (in microgram scale) using magnetic beads are currently available but those mostly use porous magnetic beads that are not recycled and are operated manually. Commercially available magnetic particles are spherical with a diameter that is equal to or greater than 1 micron and used only for a single cycle at a very small scale (e.g. Sera-Mag from GE Healthcare Life Sciences and Dynabeads from ThermoFisher Scientific). These particles are not well suited for the current invention due to their porosity, large size, and recycling capabilities.

The magnetic particles described in this invention have a high surface area to volumetric ratio. For example, the surface area to the volumetric ratio of tetrahedron and cubic particles is at least 25% greater than the ratio of spherical particles. The attachment of a ligand to the surface of magnetic particles is achieved by a linker that can bind to the ligand and the inorganic surface of magnetic particles. In one embodiment, linkers with silane groups are used to chemically bond with magnetic particles. The other end of the linker is chosen such that it can directly or indirectly bind to the ligand that is used for purification. For example, the epoxide group at the other end of the linker can react with primary amine, sulfhydryl, or carboxyl group that are commonly found on different amino acids of ligand proteins. 3-Glycidoxypropyl trimethoxysilane or 3-Glycidoxypropyl triethoxysilane are two examples of such linkers. In our studies, 0.5-3% w/w treatment with 3-Glycidoxypropyl trimethoxysilane provided excellent conjugation to Fe3O4 cubic particles of 300 nm average size.

When the donor's bodily fluids are mixed in with magnetic particles that have a surface affinity for biomaterials of interest, those biomaterials bind to the magnetic particles. Magnetic particles are captured in a separation chamber by applying an external magnetic force. Flow-through material is collected and can be infused into the donor. The captured magnetic particles can then be washed multiple times to remove impurities and then elution is performed by introducing an elution buffer that dissociates the binding of the macromolecules of interest from the magnetic particles. In one embodiment, low pH buffer (generally below pH 4.0) is used to dissociate antibodies or immunoglobulins that are captured by Protein A coupled magnetic particles. In another embodiment, low or high pH buffer (below 6.5 or higher than 8.4) is used to dissociate macromolecules bound to antibody-coupled magnetic particles. Similarly, change in pH and/or salt concentration can be used to elute factor VIII bound to quaternary ammonium particles with the anion-exchange property. If specific cells are the targeted material, the elution buffer may contain peptides or molecules that compete with interaction (between affinity magnetic particles and receptor or molecule on cell's surface) or enzymes that destabilize the interaction. The eluted biomaterials flow through and are collected or discarded (depending on the application) while the magnetic particles are retained due to the external magnetic field.

During affinity purification, the bound particles are washed either in the presence or absence of a magnetic force to remove any nonspecifically bound impurities. The bound macromolecules are eluted from the magnetic particles by introducing an elution buffer to the separation chamber in the presence or absence of an external magnetic force. The whole cycle can be repeated multiple times until the desired amount of target biomaterial is purified or removed.

If the affinity performance of particles reduces after a certain number of cycles, due to nonspecific occlusion of specific binding sites by impurities, the particles can be regenerated using regeneration solutions (e.g. solution with NaOH or chaotropic agents: Urea, Guanidine etc.).

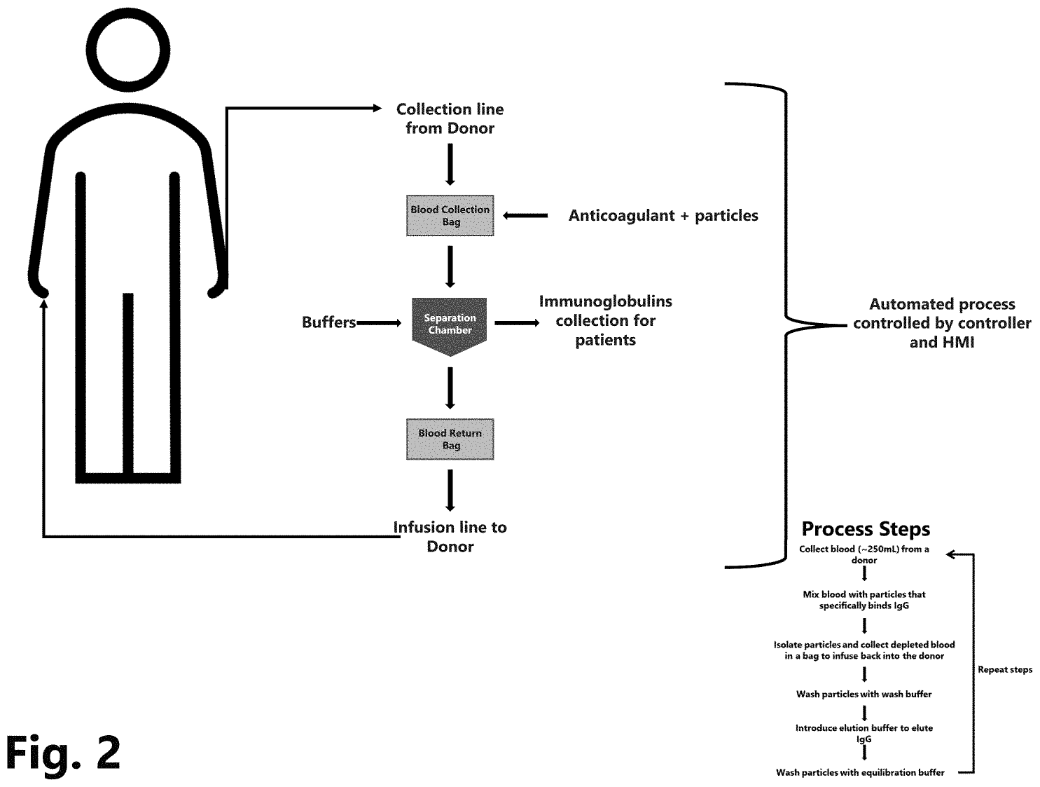

In each cycle, a specific volume of bodily fluid is collected in a bag that is attached to the system. This volume is based on guidelines for the amount of bodily fluid that can be drawn from a subject at a time. This is called the cycle volume. For example, the typical blood donation range for humans is 250-500 mL. If multiple cycles are to be performed, 250 mL of blood can be drawn per cycle (cycle volume) so that as soon as the bodily fluid donation bag is empty, another 250 mL of blood draw is initiated. Overview of the process to purify immunoglobulins from human blood is shown in FIG. 2.

Magnetic beads with affinity against target molecule and conditioning buffer or reagents (e.g. anti-clotting agent) can either be pre-loaded in the bag, or added later via another bag that can be aseptically attached, or aseptically injected via a syringe. The mixture in the bag is mixed by recirculation, impeller, or other mechanical means. The magnetic beads are recycled and their replenishment after the first cycle is generally not required. However, an anti-clotting agent or another appropriate buffer may need to be injected before every cycle of bodily fluid collection.

A cylindrical separation chamber with a conical bottom (with an apex at the bottom) is used to isolate magnetic particles during different parts of the process. In another embodiment, the top of the separation chamber is conical with apex at the top. The separation chamber is fabricated out of materials that have poor attraction for magnetic force (e.g. LDPE, high durometer Silicone, polypropylene, polystyrene, EVA, PVC etc.) and may contain steel wool or wire mesh to enhance the magnetic field via high gradient magnetic field generation, to capture magnetic particles that are small and have weak magnetic susceptibility. In one embodiment, the material of steel wool or wire mesh (1 micron-1000 micron) is ferromagnetic stainless-steel (e.g. 430 or 410 stainless steel) to provide corrosion resistance. The ferromagnetic steel wool can also be coated with metals (e.g. Nickel or Chromium) or plastics to substantially reduce or eliminate corrosion.

The external magnetic field, generally in the range of 0.1-1.7 Tesla is applied to the separation chamber by either moving permanent magnets to the proximity or turning on an electromagnet surrounding the chamber or by other means. As required in the purification process, the controller controls the magnetic force applied to the separation chamber by turning it on or off or by reducing or increasing its strength.

The complete disposable manifold comprising tubing, connectors, separation chamber (with or without steel wool), mixing bag, pH/conductivity flow cell, optical density measurement flow cell, pressure sensors, absorbance measurement flow cell, and the filter is pre-assembled and pre-sterilized prior to use. The complete assembly is designed to withstand multiple cycles in a batch. Tubing or pipes that have single-use product contact surfaces are used to circulate different fluids through the system. For example, flexible silicone, C-flex, or PVC tubing is used to connect different parts of the system to direct fluids. A disposable manifold is connected to bags or vessels containing magnetic particles, different buffers, donated bodily fluids, and final purified material via aseptic connectors or welding of a thermoplastic tube.

A controller with HMI (human machine interface) is used by the user to automatically or manually control and receive information from parts of the system, which comprise pumps (speed, direction, start/stop, feedback etc.), valves (open, close, feedback), occlusion/pressure sensors, optical density and absorbance detector, bubble sensors, in-line pH/conductivity meter, temperature sensor, weight load cells, and magnetic-field control system (FIG. 2). This information is provided as analog or digital signals to and from the controller. Commercially automation systems from various vendors such as Siemens, Beckhoff, or Allen-Bradley are available as complete solutions that include controller, input/output digital or analog cards, and HMI. Examples are Siemens SIMATIC S7-1200 kit or Beckhoff TwinCAT 3 automation platform. During the process, process data can be electronically stored or exported out (to an external data management system) by these platforms to meet CFR Part 11 compliance.

The user inputs various process parameters into a process screen that is part of the HMI. This can be turned into a recipe that is executed each time a process is run. Process parameters include cycle volume, incubation, mixing, wash, and recirculation times, pH, conductivity, and optical density and absorbance values to start or stop a step. Once the user starts the process by providing input to the HMI, the controller can follow the process screen and automatically run the process using entered process parameters. Multiple valves are controlled (FIG. 1; V1-V13) to form different fluid paths during different phases of the purification process. In one embodiment, pinch valves are used to open and close the fluid path within a flexible tube. The scheme shown in FIG. 2 is an example to introduce various fluids at different process steps during the purification process. An automated method to process bodily fluids is described below.

Process Steps

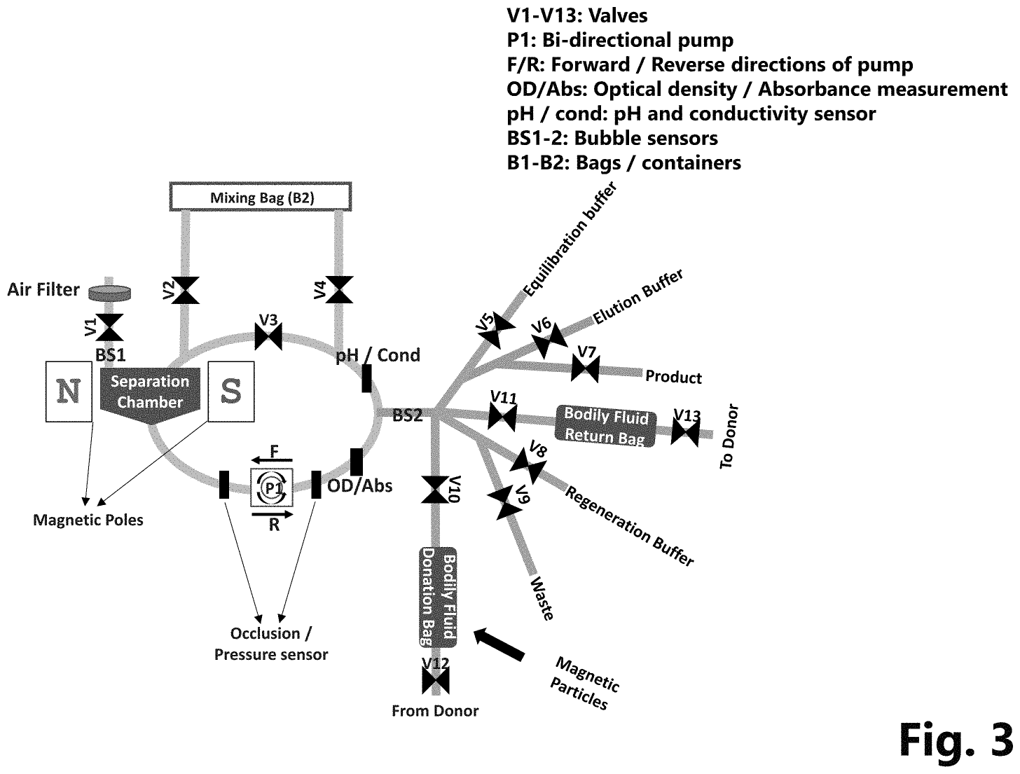

In the FIG. 3, V1-V13 are the valves, P1 indicates a bi-directional pump, FIR indicates forward/reverse directions of the pump, OD/Abs indicates Optical Density/absorbance measurement, pH/coed indicates pH and conductivity sensor, BS1-2 indicates bubble sensors, N or S indicates poles of a magnet, and B1-B2 indicates bags or containers.

The pump is turned off and all valves are closed at the end of each step. All flexible bags or containers containing different liquids are connected as per FIG. 2. A set amount of bodily fluid (cycle volume) is collected into the bodily fluid donation bag (e.g. 250 mL of blood). Sterile magnetic particles and appropriate modifiers (e.g. citrate or heparin for blood) are either injected directly in the bodily fluid donation bag or transferred through a bag containing magnetic particles. The donation and return bags are placed or hung on load cells so that accurate weight can be measured. The real-time weights information is continuously communicated to the controller. The bodily fluids start flowing into the bodily fluid donation bag once the source is connected and V12 is opened. For example, a needle for an IV tube is inserted in one arm of the donor for blood collection and the donation bag is hung at a lower height than the body. Once the cycle volume (corresponding to weight detected by the load cell) is collected, V12 is closed and Step 1 is initiated.

Step 1. Binding of Magnetic Particles to Targeted Macromolecules:

Bodily fluids are introduced into the remaining system by opening V1 and V10, turning off the magnetic field, and running the pump in the forward direction until BS1 detects liquid. At this stage, V1 is closed, V2 is opened, and the pump is continued to run in the forward direction until BS2 senses air as the bodily fluid donation bag runs empty. If the mixing bag is a rigid container (instead of a flexible bag), an air vent with a 0.2 micron filter is attached to the top of the container to displace the air by liquid coming in. Once the BS2 detects air, the V4 is opened and V10 is closed to mix magnetic beads with starting material.

If another cycle of bodily fluid donation is required, a set amount of anticoagulant or other required buffers are injected in the donation bag at this time and then V12 is opened to allow another collection of bodily fluid for the second cycle. Once the cycle volume (corresponding to weight detected by the load cell) is collected, V12 is closed.

The flow direction of the pump can be changed multiple times to improve mixing. Once the incubation time required for the binding of macromolecules to the beads is complete (typically 0.5-10 minutes), a magnetic field is turned on to capture the magnetic beads in the separation chamber. The flow rate of the pump is reduced to allow better capture of magnetic particles. The majority of the beads can be captured by this recirculation process and capture efficiency can be monitored by an optical density/absorbance sensor (e.g. monitoring OD370 for Fe3O4 particles). The pump direction is then reversed, V4 is closed, and V11 is opened to capture the remaining magnetic particles and to collect the bodily fluids (without macromolecule of interest) in the return bag. When BS2 detects air, V3 and V4 are opened and closed once to remove remnant liquid in the loop. Thereafter, V2 and V11 are closed, and the pump is stopped. After this step, V13 is opened to return the bodily fluid (without target macromolecule) to the donor. To ensure that no air can go into the return tube to the donor, V13 closes immediately once a set weight of liquid (generally 10% below the cycle volume) is infused back into the patient. To avoid hypovolemic shock, the controller is programmed such that at least 50% of the collected volume is infused back to the donor before collecting bodily fluids for the second cycle.

Step 2. Washing of Magnetic Particles with the Magnetic Field:

In this step, the magnetic field stays on and the magnetic particles are washed with an equilibration buffer to remove impurities. The equilibration buffer is chosen such that it does not disrupt the binding between the targeted macromolecule and magnetic particle (e.g. PBS or PlasmaLyte solution). After V11 is closed, the equilibration buffer is introduced into the system by opening V5 and V1 and running the pump in the forward direction until BS1 detects liquid. At that stage, V3 is opened and V5 is closed. Washing progress can be assessed by monitoring data from inline pH/conductivity sensors. After less than a minute of washing the magnetic particles in this manner, the pump direction is reversed, V9 is opened, and V3 is closed. When bubble sensor BS2 detects air, V3 is opened momentarily and then closed to allow emptying of remaining liquid in the loop. This process of washing can be repeated 1-2 times.

Step 3. Washing of Magnetic Particles without the Magnetic Field:

After V9 is closed, the magnetic field is turned off and the equilibration buffer is introduced by opening V5 and V1 and running the pump in the forward direction until 831 detects liquid. At that stage, V3 is opened and V5 is closed to generate mixing by recirculation for 0.5-2 minutes. Once the recirculation is complete, the magnetic field is turned on to collect magnetic particles in the separation chamber. The flow rate of the pump can be reduced to allow better capture of magnetic particles. Once the optical density specific to the particles (e.g. OD370) is reduced substantially, the pump direction is reversed, V9 is opened, and V3 is closed. When bubble sensor BS2 detects air, V3 is opened and closed to allow the emptying of the remaining liquid in the loop. This process of washing is repeated 1-2 times.

Step 4. Elution:

The elution buffer is used to dissociate targeted macromolecules from magnetic particles. For example, low pH buffer (100 mM Sodium Citrate buffer with pH<4) can be used to dissociate both antibody-antigen interactions as well as protein A-IgG interactions.

Step 4.1: After V9 is closed, V6 and V1 are opened, and the pump is operated momentarily in the forward direction to fill the tube up to the pump. Then V6 is closed, V9 is opened, and the pump direction is reversed. This step cleans tubes with the elution buffer.

Step 4.2: To fill the separation chamber with the elution buffer, the magnetic field is turned off and the elution buffer is introduced by opening V6 and running the pump in the forward direction until BS1 detects liquid. At that stage, V3 is opened and V6 is closed. After recirculation of the magnetic particles to create mixing, the magnetic field is turned on to collect magnetic particles in the separation chamber. The flow rate of the pump can be reduced to allow better capture of magnetic particles, Once the OD to detect particles (e.g. OD370) is reduced substantially and the OD to detect eluted molecules (e.g. OD at 280 nm for protein-based macromolecules) is maximal, V7 is opened, V3 is closed, and pump direction is reversed to run until BS2 detects air. To empty the remaining liquid in the loop, V3 is opened and closed while the pump is running and then V3 and V7 are closed. This elution process is repeated from step 4.2 for 1-2 times to collect eluted macromolecules or biomaterials. When this process is repeated, the step to clean the tubes with the elution buffer (Step 4.1) is not required.

Step 5. Washing of Magnetic Particles without the Magnetic Field:

At first, the magnetic field is turned off, the equilibration buffer is introduced by opening V5 and V1 and running the pump in the forward direction until BS1 detects liquid. At that stage, V3 is opened and V5 is closed. After recirculation of the magnetic particles to create mixing, the magnetic field is turned on to collect the magnetic particles in the separation chamber. The flow rate of the pump can be reduced to allow better capture of the magnetic particles. Once the optical density specific to the particles (e.g. OD370) is reduced substantially, the pump direction is reversed, V9 is opened, and V3 is closed. When bubble sensor BS3 detects air, V3 is opened momentarily and closed to allow emptying of remaining liquid in the loop. At last, the pump is stopped, and all valves are closed. Washing progress can be assessed by monitoring data from inline pH/conductivity sensors. This process of washing is repeated 1-2 times.

After step 5, steps 1-4 are repeated until sufficient targeted macromolecules have been purified. At that point, the last cycle is completed, and the disposable assembly can be discarded.

If the purified material is not usable (to remove autoantibodies or overproduction of cytokines etc.), it is discarded. Otherwise, the purified material is saved. If immunoglobulins are the product, the purified immunoglobulins are kept at low pH for viral inactivation and then a set amount of neutralizing buffer is added (via a syringe or aseptically connected external bag) to increase the pH to physiological range (about pH 6-8) and make the final solution isotonic. The purified macromolecules can be exposed to UV or are nanofiltered to remove or reduce contaminating virus particles. Depending on the amount of collected macromolecules, the final product can be aliquoted into several bags or vials and stored at a lower temperature. An appropriate dosage can be administered to patients in need. The purified macromolecules can also be lyophilized to increase their shelf life and to reduce transportation and logistics costs.

In addition to the applications in medical and biomanufacturing areas, there are many other industrial applications of this platform. For example, in the food and beverage industry this platform can be used to remove unwanted impurities to reduce toxicity or to enhance flavors. The device and method can be used to extract specific components that may have value. This method can also isolate components that are at very low concentration in a mixture.

### REFERENCES

1. MAbs. 2009 September-October; 1(5): 443-452.
2. Subramanian, G. (2014). Continuous Processing in Pharmaceutical Manufacturing: Wiley-VCH Verlag GmbH & Co. KGaA
3. Casadevall A, Scharff M D. Return to the past: the case for antibody-based therapies in infectious diseases. Clin Infect Dis. 1995; 21(1):150-161.
4. Nat Rev Microbiol. 2004; 2(9):695-703.
5. Gajic O, et al. Transfusion-related acute lung injury in the critically ill: prospective nested case-control study. Am J Respir Crit Care Med. 2007; 176(9):886-891.
6. https://medlineplus.gov/ency/article/000820.htm

1. A device to separate material(s) of interest from a mixture containing plurality of materials, the device comprising:
at least one disposable separation chamber with at least one inlet and one outlet;
at least one disposable bag connected by tubing to inlet and outlet;
at least one pump to move fluid in tubes;
at least one valve to direct fluid in the system;
at least one disposable connector and tubing to connect parts of the device
at least one pH, conductivity, pressure, occlusion, bubble, or optical density sensor;
a magnetic field generation unit that is controlled by a controller;
particles that: (i) are mostly non-porous (ii) are attracted to magnetic field (iii) have outer surface with affinity for material of interest;
a controller in communication with: (i) pumps to control their speed and direction, (ii) magnetic field unit to control the strength of magnetic field (iii) valves to control their open or closed state (iii) sensors to receive inputs from sensors (iv) a Human Machine Interface in which user inputs recipe parameters for a process and user can input to control pumps, valves, and magnetic field;
wherein in operation, the controller changes the state of valves and creates a fluid path through which a pump transfers a fluid to the separation chamber;
wherein in operation, the controller changes the state of valves and creates a fluid path through which a pump recirculates the fluid to provide mixing of the particles with the fluid,
wherein in operation, the controller increases the strength of the magnetic field to immobilize particles in the separation chamber,
wherein in operation, the controller changes the state of valves and creates a fluid path through which a pump transfers a fluid to a bag,
wherein in operation, the controller lowers the strength of the magnetic field to suspend particles in the separation chamber.

2. The device of claim 1 wherein the separation chamber has a conical bottom with apex at the farthest bottom.

3. The device of claim 1 wherein the size of particles is between 10-5000 nano meters.

4. The device of claim 1 wherein the particles have cubic or tetrahedron shape with average size between 10-5000 nano meters.

5. The device of claim 1 wherein at least 10 percent of the product-contact materials are disposable.

6. The device of claim 1 wherein high gradient magnetic field is created with magnetizable or ferromagnetic material in the separation chamber.

7. The device of claim 1 wherein mixing of particles with fluid is created by an impeller.

8. The device of claim 1 wherein more than one cycle of process is run without changing the product-contact material.

9. The device of claim 1 wherein all product contact surfaces are sterilized prior to use.

10. A method of using the device to separate material(s) of interest from a mixture containing plurality of materials, the method comprising:
mixing the starting material containing a mixture of materials with particles that are attracted to magnetic field and have specific affinity for a material of interest,
capturing particles in the separation chamber by changing the strength of the magnetic field and discarding or collecting the flow-through material,
washing particles with a buffer in the presence of magnetic field,
washing particles by recirculating a buffer in the absence of magnetic field and then in the presence of magnetic field to retain particles in the separation chamber,
discarding wash buffer, introducing elution buffer in the separation chamber, and mixing via pump recirculation in the absence of magnetic field followed by in the presence of magnetic field to elute the material specifically bound to the beads, and repeating this step to improve recoveries and to clean the beads.

11. The method of claim 10 wherein the starting material is unpurified or partially purified cell culture or microbial culture.

12. The method of claim 10 wherein the starting material is unpurified or partially purified bodily fluid from animals or humans.

13. The method of claim 10 wherein the beads are regenerated by exposing beads to regeneration buffer in the absence of magnetic field followed by in the presence of magnetic field to remove any non-specific impurities bound to the beads.

14. The method of claim 10 wherein the material of interest is organic or inorganic material.

15. The method of claim 10 wherein the material of interest is virus, bacteria, cell, or organelle.

16. The method of claim 10 wherein the material of interest is a protein, carbohydrate, lipid, DNA, or RNA.

17. The method of claim 10 wherein the volume of starting material is greater than 50 milliliters.

18. The method of claim 10 wherein the volume of starting material is greater than 1 Liter.
